(19) United States
(12) Patent Application Publication
(10) Pub. No.: US 2026/0283145 A1
(43) Pub. Date: Sep. 24, 2026

(54) BIOLOGICAL TISSUE STORAGE CONTAINER AND METHOD OF USING THE SAME

(71) Applicant: RM BIOSCIENCE CO., LTD., Gyeonggi-do (KR)

(72) Inventor: IL KIM, Seoul (KR)

(21) Appl. No.: 18/996,061

(22) PCT Filed: Jun. 8, 2023
(86) PCT No.: PCT/KR2023/007820

§ 371 Date: Jan. 17, 2025

(30) Foreign Application Priority Data
May 3, 2023 (KR) 10-2023-0057460

(51) Int. Cl. A01N 1/142

(52) U.S. Cl. CPC A01N 1/142

(57) ABSTRACT

A biological tissue storage container is configured for storing a biological tissue collected from an affected part while fixing the biological tissue in a preservation solution. The biological tissue storage container includes a container body, a cap member, a cutter holder, a safety ring, and a coupling unit. The biological tissue storage container may be conveniently used when accommodating a biological tissue in the storage container or when collecting the biological tissue from the storage container for tests, and may enable the protection of medical staff from the leakage of a preservation solution such as formalin during use of the storage container.

### BACKGROUND

### 1. Technical Field

The present disclosure relates to a biological tissue storage container and a method of using the same, and particularly, to a biological tissue storage container and a method of using the same, whereby the biological tissue storage container may be conveniently used when accommodating a biological tissue in the storage container or when collecting the biological tissue from the storage container for tests, and may enable the protection of medical staff from the leakage of a preservation solution such as formalin during use of the storage container.

In addition, the present disclosure relates to a biological tissue storage container and a method of using the same, whereby the use time of a preservation container by medical staff may be shortened by smoothing a cutting part of a sealing film and forming an auxiliary through hole to make the movement speed of the preservation solution faster.

### 2. Background Art

For example, tissues collected from affected areas for tests are stored in a storage container containing formalin, which is a preservation solution, and tissue testers take out biological tissues of a microscopic size from the storage container for tests.

When putting the collected affected tissues in the storage container or taking the collected affected tissues out for tests, care needs to be taken to prevent medical staff such as doctors and nurses from being directly exposed to formalin which is harmful to the human body.

Korean Patent Registration No. 10-2069602 discloses an example of an affected tissue storage container which is shown in FIGS. 1 and 2. In the affected tissue storage container, a skirt portion 44 is integrally connected and coupled to a lid 40A by a connecting piece 46, and the preservation solution L is accommodated in an inner cylinder 50A and supplied in a sealed state by a sealing tape 51A.

The storage container is used by breaking the connecting piece 46 and removing the skirt portion 44. When the skirt portion 44 is removed and the lid 40A is screw-rotated and lowered, the sealing tape 51A is punched by a cutter 36, and the preservation solution L accommodated in the inner cylinder 50A is moved to an outer cylinder 20A accommodating an affected tissue M to preserve the affected tissue.

When collecting the affected tissue M for tests in the laboratory, as shown in FIG. 3A, the storage container is turned upside down so that the storage solution L accommodated in the outer cylinder 20A is moved back to the inner cylinder 50A to be accommodated. At this time, the affected tissue M is filtered by a mesh 32 of an inner cap 30A.

Next, as shown in FIG. 3B, when the outer cylinder 20A is separated, the affected tissue may be picked up by tweezers, etc.

However, the storage container as described above has the following problems.

First, because the storage container has a structure in which the skirt portion 44 is integrally connected to the lid 40A by the connecting piece 46, there is a risk of human damage when the connecting piece 46 breaks and the structure is complicated, making it difficult to manufacture.

Second, a process of accommodating the affected tissue M in the outer cylinder 20A need to break the connecting piece 46 and separate the skirt portion 44 separated from the lid 40A from the outer cylinder 20A, making it inconvenient to use.

That is, there is the inconvenience of having to sequentially separating the lid 40A and the inner cap 30A to which the inner cylinder 50A is coupled from the outer cylinder 20A.

Third, when the lid 40A is lowered while rotating, the sealing tape 51A is punched by the cutter 36, and after punching, the inner cylinder 50A and the inner cap 30A is rotated together using the sealing tape 51A as a connecting link, and thus the sealing tape 51A is punched, but there is a problem in that the movement speed of the preservation solution L is low due to the lack of tearing.

Fourth, as shown in FIGS. 3A and 3B, when the preservation solution L is moved to the inner cylinder 50A by turning a transport container upside down, the preservation solution L flows to the space between the inner cylinder 50A and the lid 40A through a gap between the inner cap 30A and the outer cylinder 20A, and there is a risk that the tester is exposed to harmful drugs such as formalin.

### SUMMARY

The present disclosure is directed to providing a biological tissue storage container and a method of using the same, whereby the biological tissue storage container may be conveniently used when accommodating a biological tissue in the storage container or when taking the biological tissue out of the storage container for tests.

In addition, the present disclosure is directed to providing a biological tissue storage container and a method of using the same, which enable the protection of medical staff from the leakage of a preservation solution such as formalin during use of the storage container.

In addition, the present disclosure is directed to providing a biological tissue storage container and a method of using the same, whereby the use time of the storage container by medical staff may be shortened by making the movement speed of the preservation solution faster.

According to the present disclosure, there is provided a biological tissue storage container for storing a biological tissue collected from an affected part while fixing the biological tissue in a preservation solution including a container body having an open upper portion and a closed lower portion, a first screw portion formed on an upper outer circumferential surface, a support projection formed on a lower side of the first screw portion, and a tissue accommodating portion formed to accommodate the biological tissue; a cap member including an upper surface portion, a fastening cap extending downward from an edge of the upper surface portion and having a second screw portion formed on an inner circumferential surface and screw-coupled to the first screw portion, an accommodation cap extending downward from the upper surface portion concentrically with the fastening cap on the inside of the fastening cap to form an accommodation space accommodating the preservation solution, enabling entry into the inside of the container body, and having an open lower end, an annular space formed between the fastening cap and the accommodation cap and into which a holder body enters, and a sealing film that is breakable and adhered to a lower end of the accommodation cap to seal the accommodation space; a cutter holder having the cylindrical holder body to which the accommodation cap is coupled inward, a bottom surface disposed at a lower end of the holder body and having a plurality of through holes formed therein, and a cutter formed on an upper surface of the bottom surface to break the sealing film; a cylindrical safety ring supported by the support projection, detachably coupled to the container body, and preventing breakage of the sealing film by the cutter by preventing the cap member from lowering; and a coupling means connecting the accommodation cap to the holder body to have a first frictional force to prevent the cutter holder from freely falling from the accommodation cap, and at the same time, a second frictional force to enable relative movement of the accommodation cap with respect to the holder body.

In addition, in the storage container of the present disclosure, at least one of the accommodation cap and the holder body may include a synthetic resin material capable of elastic deformation, and a plurality of ribs may be formed on an outer circumferential surface of the accommodation cap to protrude long vertically at regular intervals and at the same height, so that the accommodation cap and the holder body may be fitted and coupled by the plurality of ribs.

In addition, in the storage container of the present disclosure, the sealing film may include a vinyl film heat-sealed at a lower end of the accommodation cap and a metal thin film bonded to the vinyl film, a diameter of the sealing film may be relatively larger than an outer diameter of the accommodation cap, so that a skirt portion protruding to the outside of the accommodation cap is formed, and when the accommodation cap and the holder body are coupled to each other, the skirt portion may seal between the accommodation cap and the holder body to prevent the preservation solution from leaking into the annular space.

In addition, in the storage container of the present disclosure, in the cutter holder, the cutter may be formed in plural and arranged concentrically at the same height, and a center post relatively higher than a height of each cutter may be formed in the center of the bottom surface, and a perimeter space into which the accommodation cap enters may be formed between each cutter and the holder body, so that the sealing film may be tightened by the center post just before the sealing film is broken by each cutter.

In addition, in the storage container of the present disclosure, a plurality of auxiliary through holes may be formed in a bottom surface of the perimeter space.

In addition, in the storage container of the present disclosure, a plurality of first locking protrusions may be formed on the bottom surface of the cutter holder on the same circumference, an annular projection may be disposed on an upper side of the tissue accommodating portion, protrude from an inner wall surface of the container body, and support the plurality of the first locking protrusions, and at least one first stopper may be formed to protrude on an upper surface of the annular projection such that each of the first locking protrusions may be locked.

In addition, in the storage container of the present disclosure, a plurality of second protrusions may be formed to be spaced inward from an edge of the bottom surface of the cutter holder and disposed on the same circumference, an annular seating projection may be disposed on an upper side of the tissue accommodation portion to protrude from the inner wall surface of the container body, and on which a seating portion at an edge position of the bottom surface of the cutter holder may be slidably seated, and a plurality of second stoppers in which the second locking protrusions are locked may be formed on the side of the annular seating projection at regular intervals.

In addition, in the storage container of the present disclosure, an annular packing groove may be formed in the seating portion of the bottom surface of the cutter holder, and a packing ring may be coupled to the packing groove so as to be coplanar with the bottom surface of the cutter holder.

According to the present disclosure, there is provided a method of using a biological tissue storage container including,
Biological tissue accommodation step
   a first step, performed by a user, of holding the container body with one hand, holding the cap member with the other hand, and rotating and separating the cap member from the container body, wherein the cutter holder and the cap member while being coupled by the coupling means are separated from each other; a second step of placing the cutter holder and the cap member separated from the container body on a floor; a third step of separating the safety ring from the container body with a thumb and index finger of the hand holding the container body; a fourth step of accommodating the biological tissue collected from an affected part with the hand that held the cap member in the container body; a fifth step of while holding the container body with one hand, screw-coupling the cap member and cutter holder to the container body with the other hand, breaking the sealing film with the cutter, and moving the preservation solution to the tissue accommodation portion in which the biological tissue is accommodated;
Biological tissue collection step
   a sixth step of, after the fifth step, erecting the container body so that the cap member is in contact with the floor, moving the preservation solution of the tissue accommodation portion to an accommodation space of the cap member through the cutter holder, and then allowing the biological tissue to remain on the bottom surface of the cutter holder; a seventh step of separating the container body from the cap member by holding the cap member with one hand, and rotating the container body with the other hand; and an eighth step of holding the cap member with one hand, and separating the biological tissue from the cutter holder using tweezers with the other hand.

According to the present disclosure, there is provided a method of using a biological tissue storage container including,
Biological tissue accommodation step
   a first step, performed by a user, of holding the container body with one hand, holding the cap member with the other hand, and rotating and separating the cap member from the container body, wherein the cutter holder and the cap member while being coupled by the coupling means are separated from each other; a second step of placing the cutter holder and the cap member separated from the container body on a floor; a third step of separating the safety ring from the container body with a thumb and index finger of the hand holding the container body; a fourth step of accommodating the biological tissue collected from an affected part with the hand that held the cap member in the container body; a fifth step of while holding the container body with one hand, screw-coupling the cap member and cutter holder to the container body with the other hand, breaking the sealing film with the cutter, and moving the preservation solution to the tissue accommodation portion in which the biological tissue is accommodated;
Biological tissue collection step
   a sixth step of, after the fifth step, erecting the container body so that the cap member is in contact with the floor, moving the preservation solution of the tissue accommodation portion to an accommodation space of the cap member through the cutter holder, and then allowing the biological tissue to remain on the bottom surface of the cutter holder; a seventh step of separating the container body from the cap member by holding the cap member with one hand, and rotating the container body with the other hand; and an eighth step of holding the cap member with one hand, and separating the biological tissue from the cutter holder using tweezers with the other hand.

First, the storage container 100 of the embodiment of the present disclosure has a structure in which the cap member 20 and the cutter holder 30 maintain a coupled state and are coupled to enable relative movement to each other, and thus the number of actions of a user may be reduced when accommodating the tissue T in the container body 10 or collecting the tissue T, thereby providing convenience of use and shortening the use time.

Second, unlike the related art, the storage container of the embodiment of the present disclosure is configured in which the safety ring 40 is separated from the cap member 20, which enables to separate the safety ring 40 with one hand while holding the container body 10 with one hand, making it convenient to use.

Third, despite the formation of the second stopper 17 for stopping rotation on the bottom surface 32 of the cutter holder 30, it is possible to form the seating portion 32d that is seated in surface contact with the seating projection 16 of the container body 10, thereby simultaneously ensuring to satisfy the smooth cutting of the sealing film 29 and the leakage of the preservation solution S.

Fourth, the storage container 100 of the embodiment of the present disclosure, in which the auxiliary through hole 32b is formed in the cutter holder 30, and the center post 34 is formed higher than the height of the cutter 33, may smooth the cut surface of the sealing film 29 and form the auxiliary through hole 32b to make the movement speed of the preservation solution S faster.

Fifth, the storage container 100 forms the skirt portion 29c on the edge of the sealing film 29 to allow the skirt portion 29c to be inserted between the accommodation cap 24 and the holder body 31, and thus the cap member 20 and the cutter holder 30 may be coupled to each other to be movable together and enable relative movement. In addition, the skirt portion 29c seals between the accommodation cap 24 and the holder body 31 to prevent the preservation solution S from leaking into the annular space 28, thereby protecting medical staff.

### BRIEF DESCRIPTION OF THE DRAWINGS

FIGS. 1 to 3B are cross-sectional views explaining the operation state of a storage container of the related art,

FIG. 4 is a perspective view showing a storage container of the embodiment of the present disclosure,

FIG. 5 is an exploded perspective view of FIG. 4,

FIG. 6 is an exploded cross-sectional view of FIG. 4,

FIG. 7 is a half cross-sectional perspective view of FIG. 5,

FIG. 8 is a bottom perspective view of a cutter holder,

FIG. 9 is a cross-sectional view of FIG. 4,

FIGS. 10A to 10C are diagrams showing the operation state of a storage container according to a first embodiment of the present disclosure,

FIGS. 11A to 11C are diagrams showing the operation state of a storage container according to a second embodiment of the present disclosure,

FIG. 12 is a perspective view showing another example of a cutter holder,

FIG. 13 is a main diagram showing a modified example of a container body, and

FIGS. 14A to 14C are diagrams showing the operation state of a storage container of a third embodiment of present disclosure.

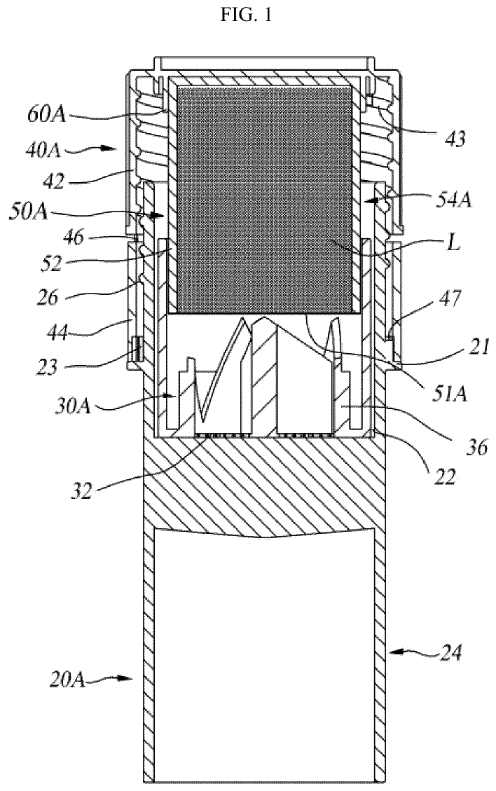

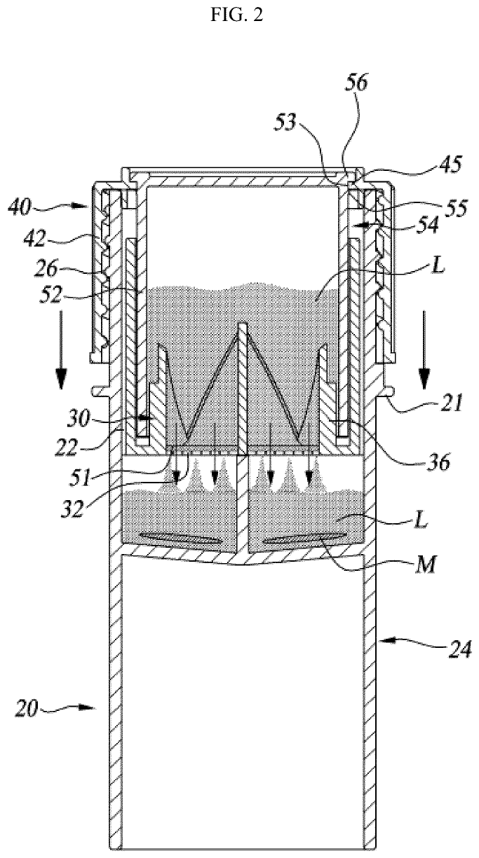

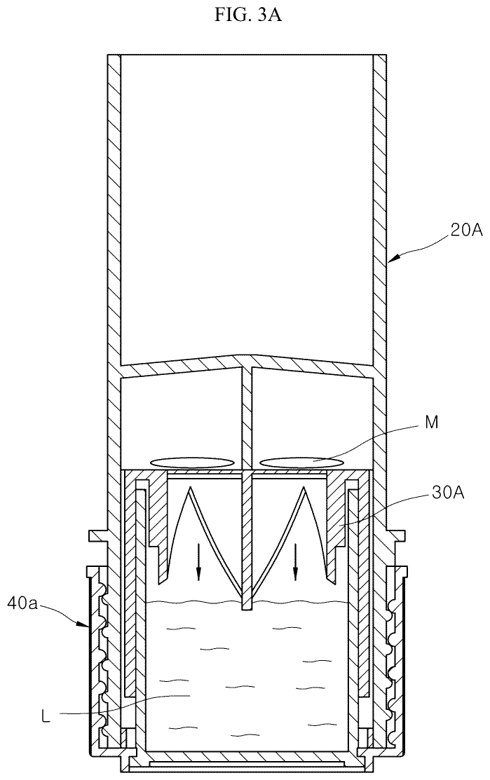

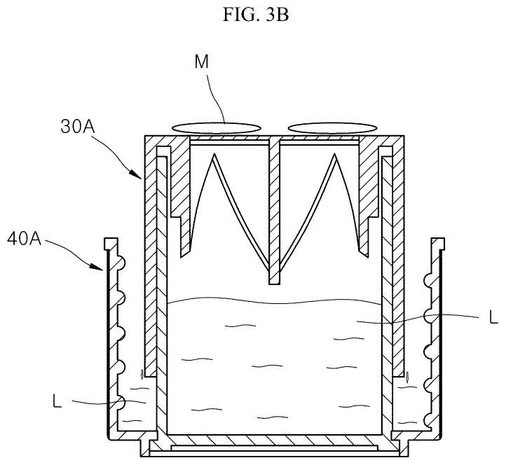

### DETAILED DESCRIPTION

According to a biological tissue storage container and a method of using the same of an embodiment of the present disclosure, the biological tissue storage container may be conveniently used when accommodating and collecting an affected tissue, medical staff is protected by preventing the leakage of a preservation solution such as formalin, and the use time of a preservation container may be shortened by making the movement speed of a preservation solution faster.

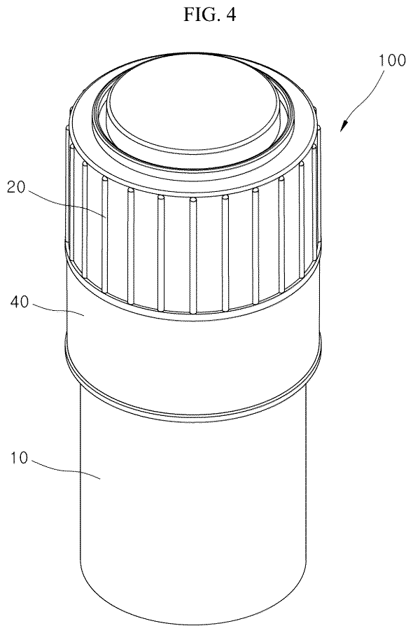

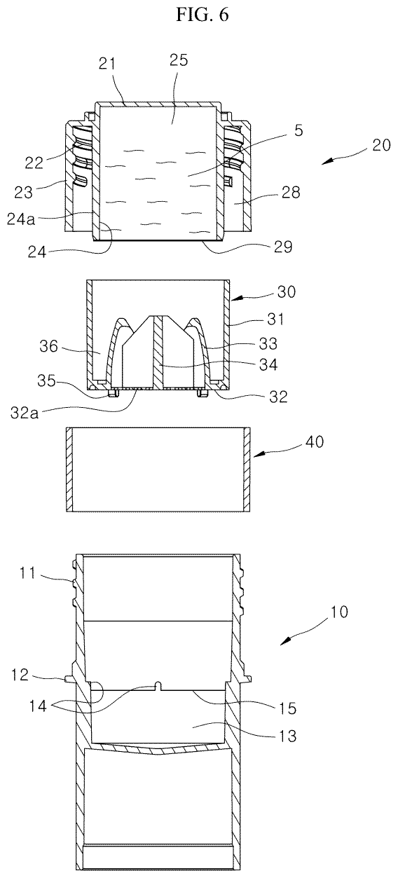

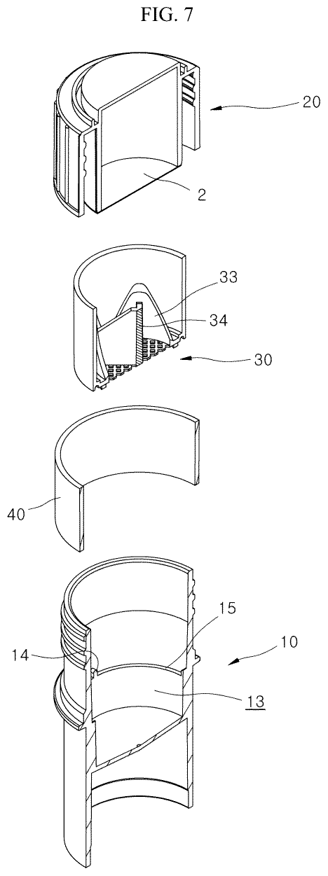

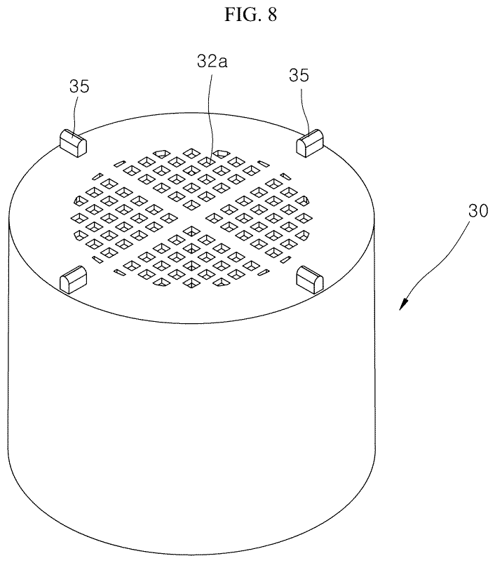

Referring to FIGS. 4 to 9, a biological tissue storage container 100 according to an embodiment of the present disclosure includes a container body 10, a cap member 20 accommodating a preservation solution S, a cutter holder 30, and a safety ring 40.

The container body 10 has an open upper portion and a closed lower portion, a first screw portion 11 formed on an upper outer circumferential surface, a support projection 12 formed on a lower side of the first screw portion 11, and a tissue accommodating portion 13 formed to accommodate a biological tissue T collected from an affected part.

The tissue accommodating portion 13 is formed in a middle part of the container body 10 to facilitate the accommodation of the biological tissue T, and is also formed long in a longitudinal direction to facilitate the user's grip.

The cap member 20 is detachably screw-coupled to the container body 10.

The cap member 20 includes an upper surface portion 21, a fastening cap 23 extending downward from the edge of the upper surface portion 21 and having a second screw portion 22 formed on an inner circumferential surface and screw-coupled to the first screw portion 11, an accommodation cap 24 extending downward from the upper surface portion 21 concentrically with the fastening cap 23 on the inside of the fastening cap 23 to form an accommodation space 25 accommodating the preservation solution S, enabling entry into the inside of the container body 10, and having an open lower end, an annular space 28 formed between the fastening cap 23 and the accommodation cap 24 and into which the holder body 31 enters, and a sealing film 29 that is breakable and adhered to the lower end of the accommodation cap 24 to seal the accommodation space 25.

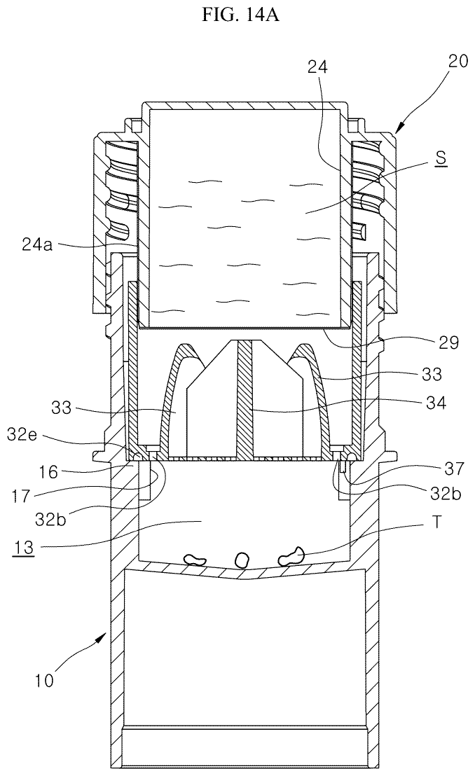

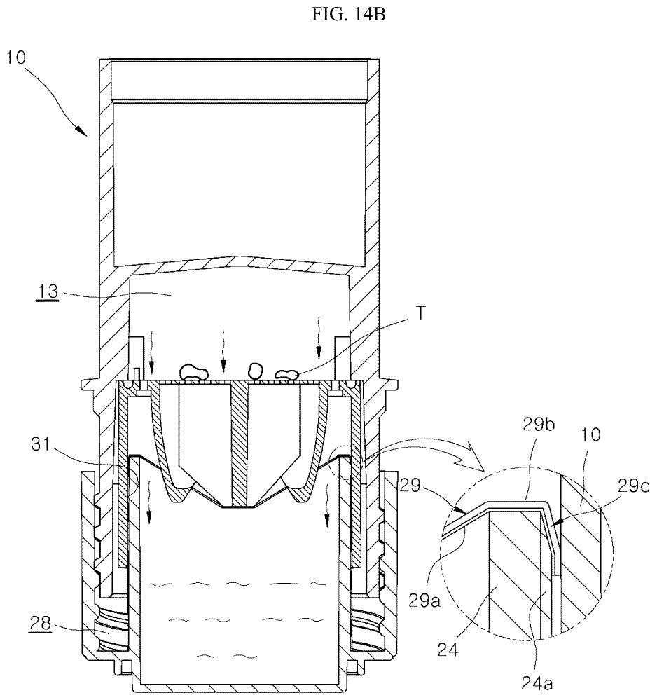

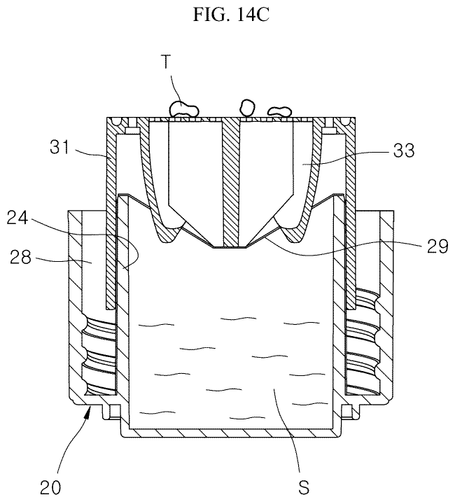

Referring to FIG. 14B, the sealing film 29 includes a vinyl film 29a heat-sealed at the lower end of the accommodation cap 24 and a metal thin film 29b such as silver foil bonded to the vinyl film 29a, and may be punched or cut by a cutter 33 to be described below.

Because the sealing film 29 includes the vinyl film 29a, when the cutter 33 is blunt or the sealing film 29 is not tight, the sealing film 29 tends to be pushed and cut during cutting, and a cut part is not smooth, which may slow down the flow speed of the preservation solution S.

The cutter holder 30 may enter and exit the container body 10, and may be coupled to and separated from the cap member 20.

The cutter holder 30 has the cylindrical holder body 31 to which the accommodation cap 20 is coupled inward, a bottom surface 32 disposed at a lower end of the holder body 31 and having a plurality of through holes 32a formed therein, and the cutter 33 formed on an upper surface of the bottom surface 32 to break the sealing film 29.

The cutter 33 is formed in plural and arranged concentrically at the same height, and each cutter 33 has a sharp upper end and side edge and is formed in an overall triangular shape.

In addition, a center post 34 of the same height as each cutter 33 is formed in the center of the bottom surface 32, and a perimeter space 36 into which the accommodation cap 24 enters is formed between each cutter 33 and the holder body 31.

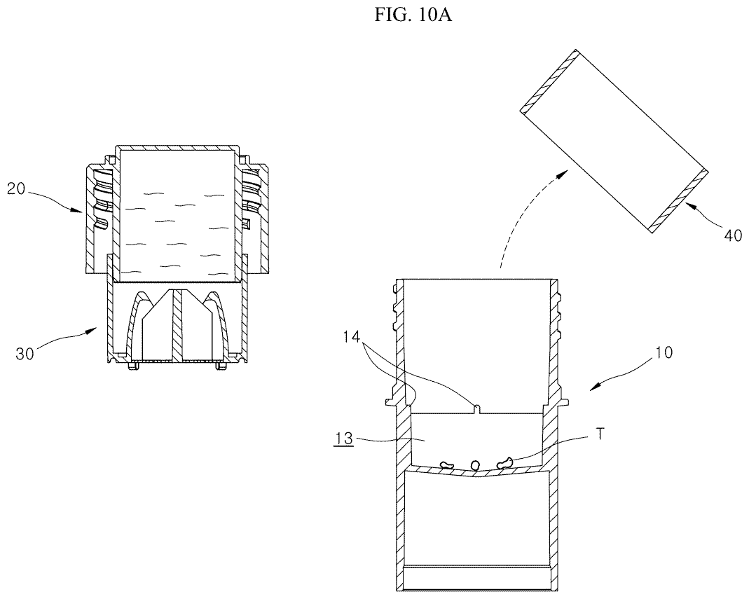

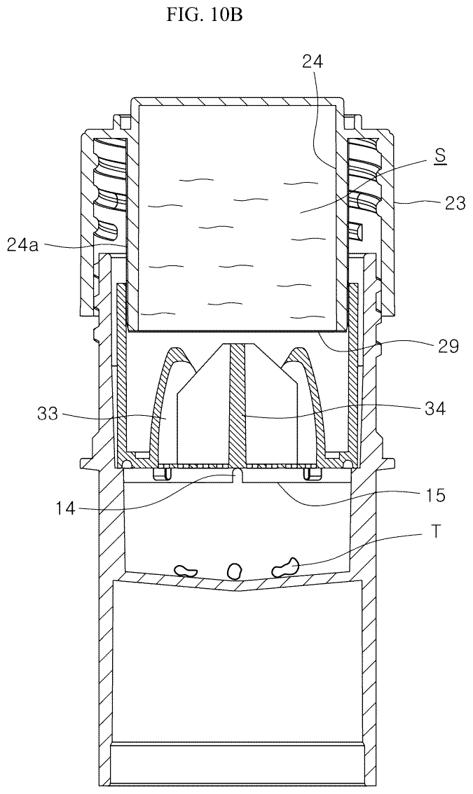

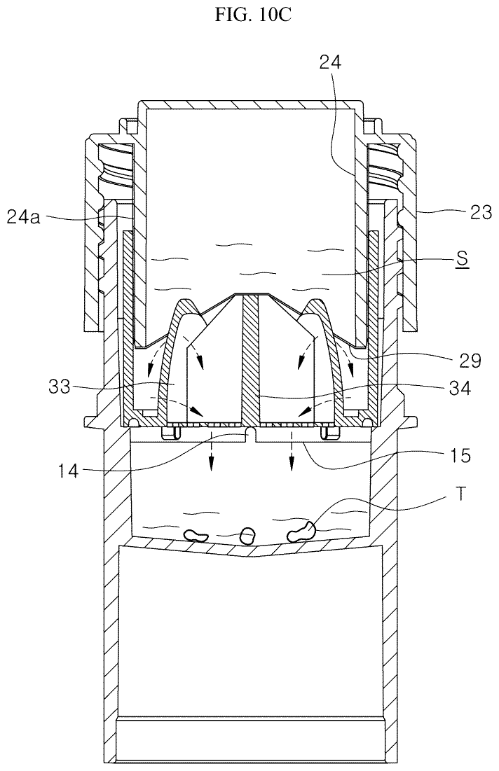

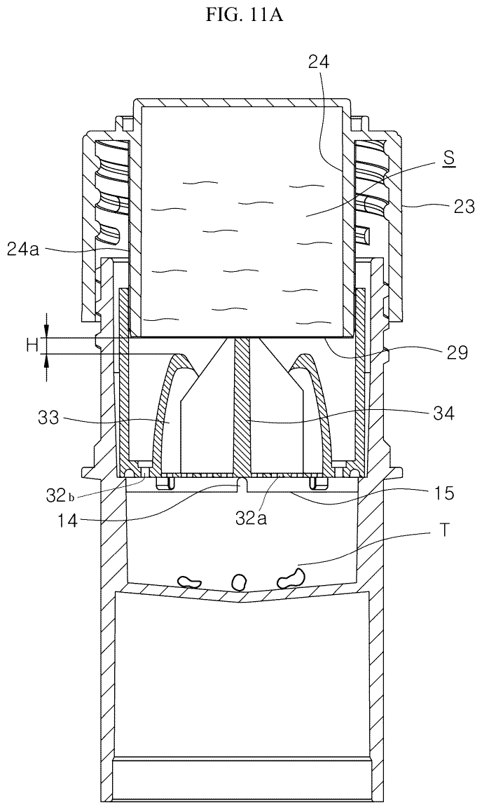

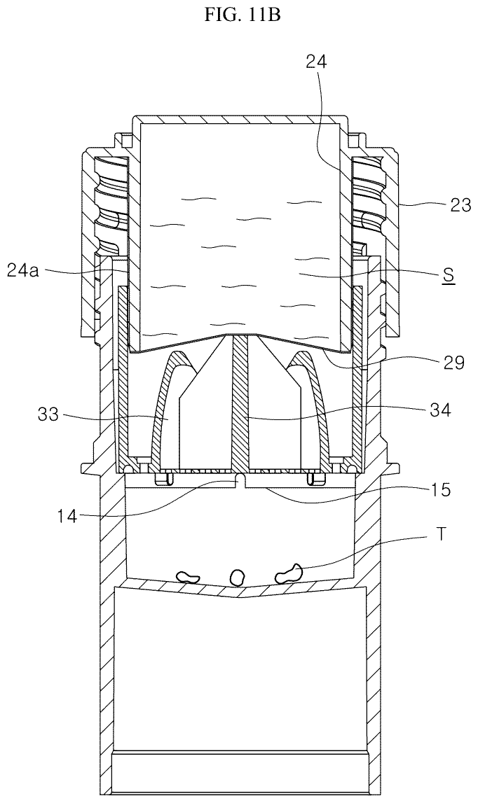

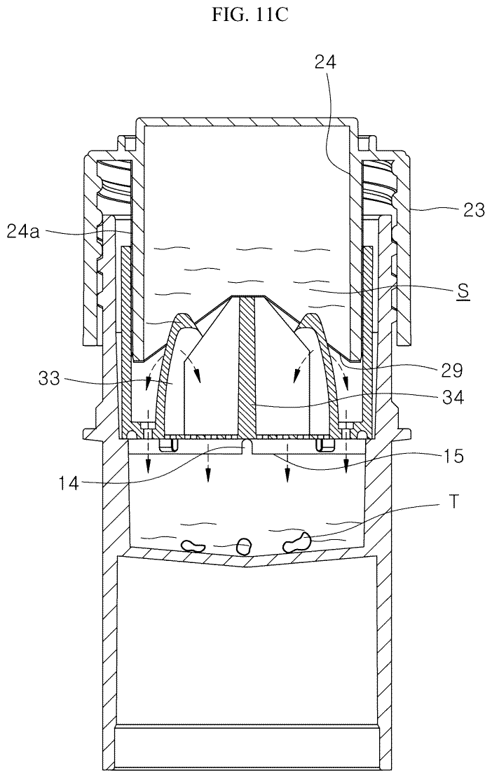

Referring to FIGS. 10C and 11C, because the final shape of the sealing film 29 becomes a cap shape after being punched and cut, the preservation solution S in the accommodation space 25 falls along an inclined surface of the sealing film 29, partially flows into the perimeter space 36, and is partially discharged through the cut part into the through hole 32a. The preservative solution S flown into the perimeter space 36 is accommodated into the tissue accommodation portion 13 through the through hole 32a beyond between the cutters 33.

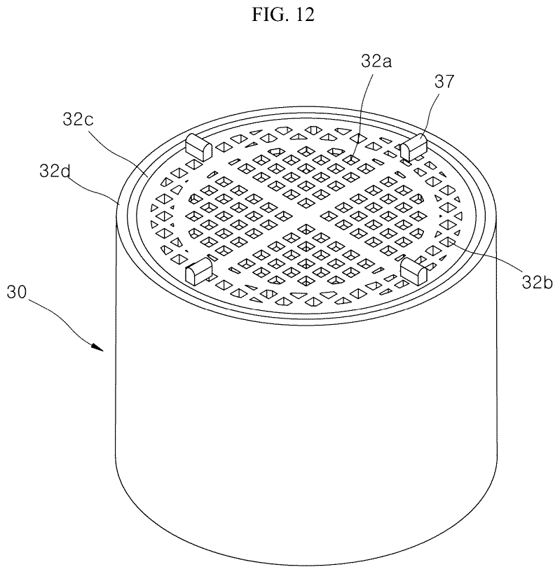

Therefore, as shown in FIGS. 11A and 12, it is preferable to shorten the movement time of the preservation solution S by forming an auxiliary through hole 32b in a bottom surface of the perimeter space 36.

Referring to FIGS. 11A and 11B showing other examples, the center post 34 is formed relatively higher than the height of each cutter 33 to have a height difference H.

This is so that the sealing film 29 is tightened by the center post 34 just before the sealing film 29 is cut by each cutter 33 when the cap member 20 is lowered. In this case, punching and cutting by the cutter 33 are performed while the sealing film 29 is tightened, and thus a cut surface is smooth and easy cutting is performed.

When the cut surface of the sealing film 29 is smooth, the downward flow of the preservation solution S may be smooth, which may improve the flow speed so as to shorten the use time of the preservation container 100.

The safety ring 40 is formed in a cylindrical shape, supported by the support projection 12 formed on the circumferential surface of the container body 10, and detachably connected to the container body 10.

Before using the storage container 100, the cap member 20 is prevented from lowering by the safety ring 40, thereby preventing breakage of the sealing film 29 by the cutter 33.

Meanwhile, in the embodiment of the present disclosure, a coupling means connecting the accommodation cap 24 to the holder body 31 is provided.

The coupling means is configured to have a first frictional force to prevent the cutter holder 30 from freely falling from the accommodation cap 24, and at the same time, a second frictional force to enable relative movement of the accommodation cap 24 with respect to the holder body 31.

In an embodiment, the coupling means is configured such that at least one of the accommodation cap 24 and the holder body 31 includes a synthetic resin material capable of elastic deformation, and a plurality of ribs 24a are formed on an outer circumferential surface of the accommodation cap 24 to protrude long vertically at regular intervals and at the same height, so that the accommodation cap 24 and the holder body 31 are fitted and coupled by the plurality of ribs 24a.

Accordingly, free fall of the cutter holder 30 from the accommodation cap 24 is prevented, the accommodation cap 24 and the holder body 31 may be rotated together, and the relative rotation of the accommodation cap 24 with respect to the holder body 31 is also possible.

The coupling means of another example is configured as follows.

Referring to the enlarged part of FIG. 14B, the diameter of the sealing film 29 is relatively larger than the outer diameter of the accommodation cap 24, and thus the sealing film 29 includes a skirt portion 29c protruding to the outside of the accommodation cap 24.

In this case, when the accommodation cap 24 and the holder body 31 are coupled to each other, the skirt portion 29c of the sealing film 29 is fitted between the accommodation cap 24 and the holder body 31.

Accordingly, free fall of the cutter holder 30 from the accommodation cap 24 is prevented without forming the plurality of ribs 24a, and the accommodation cap 24 and the holder body 31 are coupled to rotate together, and relative rotation of the accommodation cap 24 with respect to the holder body 31 is also possible.

In addition, the skirt portion 29c seals between the accommodation cap 24 and the holder body 31 to prevent the preservation solution S from leaking into the annular space 28.

The coupling means may include the plurality of ribs 24a and the skirt portion 29c together.

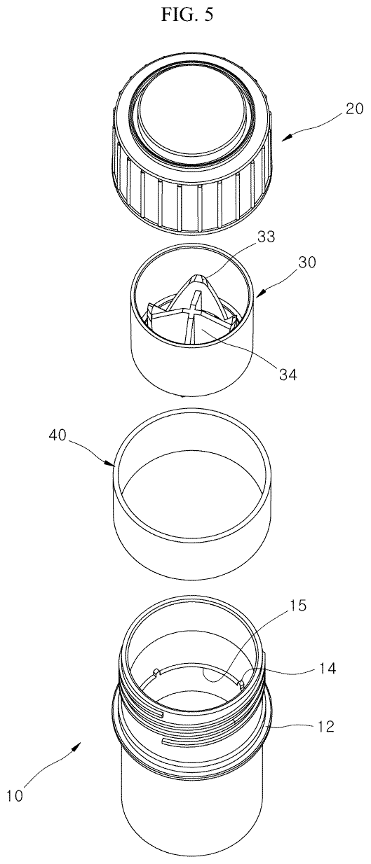

Referring to FIGS. 5, 6 and 8, a plurality of first locking protrusions 35 are formed on the bottom surface 32 of the cutter holder 30 on the same circumference at regular intervals.

An annular projection 15 is formed on an inner wall surface of the container body 10.

The annular projection 15 is disposed on an upper side of the tissue accommodating portion 13 and protrudes from the inner wall surface of the container body 10, and the first locking protrusions 35 of the cutter holder 30 are supported on the annular projection 15.

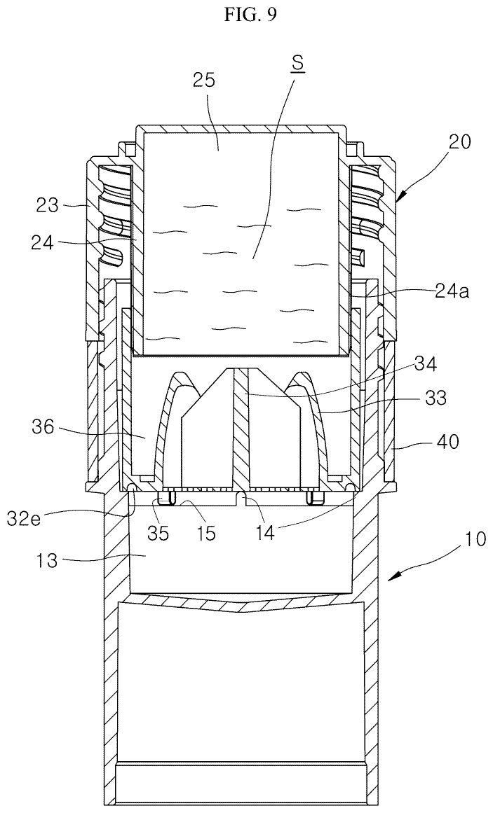

Accordingly, as shown in FIG. 9, a space of the tissue accommodating portion 13 may be formed in a lower portion of the cutter holder 30.

In addition, a plurality of first stoppers 14 are formed to protrude at regular intervals on an upper surface of the annular projection 15.

When the cutter holder 30 is rotated while being supported by the annular projection 15, each of the first locking protrusions 35 is locked in each of the first stoppers 14, and thus the cutter holder 30 is stopped rotating.

The above configuration enables the accommodation cap 24 to be rotated and moved downward while generating the second frictional force between the outer circumferential surface of the accommodation cap 24 and the inner circumferential surface of the holder body 31 when the cutter holder 30 is stopped rotating.

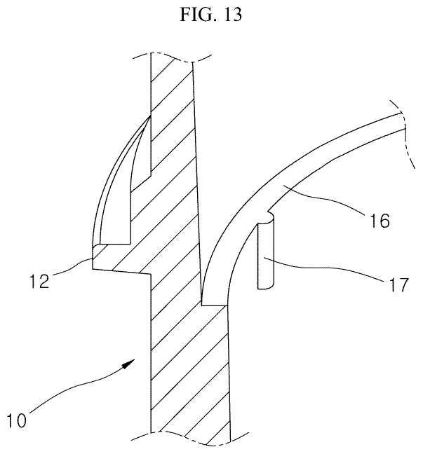

Therefore, the fastening cap 23 is rotated downward when the cutter holder 30 is stopped rotating, and thus punching and cutting of the sealing layer 29 are performed smoothly. FIGS. 12 to 14C show coupling structures of other examples of the container body 10 and the cutter holder 30.

A plurality of second protrusions 37 are formed to be spaced inward from an edge of the bottom surface 32 of the cutter holder 30 and disposed on the same circumference.

Accordingly, a flat seating portion 32d is formed at the edge of the bottom surface 32 of the cutter holder 30.

A seating projection 16 is formed on the inner wall surface of the container body 10 to be supported while being in surface contact with the seating surface 32d of the cutter holder

An annular packing groove 32c is formed in the seating portion 32d of the cutter holder 30, and a packing ring 32e is coupled to the packing groove 32c so as to be coplanar with the bottom surface 32 of the cutter holder 30. At this time, each of the packing groove 32c and the packing ring 32e has a semicircular cross-section. A planar part of the packing ring 32e preferably protrudes finely from the seating portion 32d of the cutter holder 30.

In this case, as shown in FIG. 14B, when the storage container 100 is turned upside down to collect the tissue T, the leakage of the preservation solution S between the outer circumferential surface of the cutter body 31 and the inner circumferential surface of the container body 10 is minimized, thereby protecting medical staff.

The seating projection 16 is disposed on the upper side of the tissue accommodation portion 13 and is formed to protrude from the inner wall surface of the container body 10.

In addition, a plurality of second stoppers 17 are formed to protrude from the side of the seating projection 16 at regular intervals.

When the cutter holder 30 is rotated while being supported by the seating projection 16, each of the second locking protrusions 37 are locked in each of the second stoppers 17, and thus the cutter holder 30 is stopped rotating.

The above configuration enables the accommodation cap 24 to be rotated and moved downward while generating the second frictional force between the outer circumferential surface of the accommodation cap 24 and the inner circumferential surface of the holder body 31 when the cutter holder 30 is stopped rotating.

Therefore, the cutter holder 30 is stopped rotating when the fastening cap 23 is rotated downward, and thus punching and cutting of the sealing layer 29 are performed smoothly.

Despite the formation of the second stopper 17 for stopping rotation on the bottom surface 32 of the cutter holder 30, the above configuration may form the seating portion 32d that is seated in surface contact with the seating projection 16 of the container body 10, thereby simultaneously ensuring to satisfy the smooth cutting of the sealing film 29 and the leakage of the preservation solution S.

Hereinafter, an operation of the storage container having the above configuration and a method of using the storage container will be described.
Tissue Accommodation Step

Referring to FIGS. 4 and 10A to 10C, a user (medical staff) holds the container body 10 with one hand, holds the cap member 20 with the other hand, and screw-rotates and separates the cap member 20 from the container body 10.

At this time, the cutter holder 30 and the cap member 20 are separated from each other at once while being coupled by the coupling means (rib 24a).

The cutter holder 30 and the cap member 20 separated from the container body 10 are placed on the floor, and while holding the container body 10, the safety ring 40 is separated from the container body 10 with the thumb and index finger of the hand holding the container body 10.

Unlike the related art described above, the safety ring 40 of the embodiment of the present disclosure is configured to be separate from the cap member 20, which enables separation of the safety ring 40 with one hand while holding the container body 10 with one hand, making it convenient to use.

The tissue T collected from the affected part with the hand that held the cap member 20 is accommodated in the tissue accommodation portion 13 of the container body 10.

While holding the container body 10 with one hand, the cap member 20 and cutter holder 30 are screw-coupled to the container body 10 with the other hand (FIG. 10B).

When the cap member 20 is gradually rotated downward, the cutter holder 30 is also rotated downward and moved downward together, and while the first locking protrusion 35 of the cutter holder 30 is supported by the annular projection 15, the cutter holder 30 is stopped moving downward.

In addition, when the cap member 20 is continuously rotated and moved downward even while the downward movement of the cutter holder 30 is stopped, the cutter holder 30 is temporarily rotated with the cap member 20 and the first locking protrusion 35 is locked in the first stopper 14 such that the rotation of the cutter holder 30 is stopped.

As shown in FIG. 10C, when the cap member 20 moves downward while the rotation of the cutter holder 30 is stopped, the sealing film 29 is cut by the cutter 33.

At this time, the edge of the sealing membrane 29 is cut by each cutter 33, and a central part thereof is supported by the center post 34 to be deformed into a cap shape as a whole.

Accordingly, because the sealing film 29 is deformed to have an inclined part, the preservation solution S of the accommodation space 25 flows down toward the edge of the sealing film 29, collected into the perimeter space 36 of the cutter holder 30, and then accommodated into the tissue accommodation portion 13 in which the tissue T is accommodated through the through hole 32a of the bottom surface 32.

As described above, the tissue T and the preservation solution S are accommodated together in the tissue accommodation portion 13 and stored in a sealed state with the cap member 20.
Tissue collection step

A use operation of collecting tissue T from the tissue accommodation portion 13 to test the tissue T in the laboratory is as follows.

First, the container body 10 is erected so that the cap member 20 is in contact with the floor, and the preservation solution S of the tissue accommodation portion 13 is moved to the accommodation space 25 of the cap member 20 through the through hole 32a of the cutter holder 30.

After the preservation solution S is moved, the tissue T remains on the bottom surface 32 of the cutter holder 30.

Then, the container body 10 is separated from the cap member 20 by holding the cap member 20 on the lower side with one hand, and rotating the container body 10 on the upper side with the other hand.

At this time, the cutter holder 30 maintains a stable posture while being coupled to the cap member 20.

Then, the cap member 20 is held with one hand, and the tissue T is separated from the cutter holder 30 using tweezers with the other hand.

Because the storage container 100 as described above has a structure in which the cap member 20 and the cutter holder 30 maintain a coupled state and are coupled to enable relative movement to each other, and thus the number of actions of the user may be reduced when accommodating the tissue T in the container body 10 or collecting the tissue T, thereby providing convenience of use and shortening the use time.

FIGS. 11A to 11C show operating states of the storage container 100 in which an auxiliary through hole 32b is formed in the cutter holder 30 and the center post 34 is formed higher than the height of the cutter 33. Other configurations are the same as above, and thus descriptions of the same parts will be omitted.

Immediately before the sealing film 29 is cut by each cutter 33 when the cap member 20 is lowered, as shown in FIG. 11B, the sealing film 29 is first pressed against the center post 34, so that the sealing film 29 is tightened.

In this case, punching and cutting by the cutter 33 are performed while the sealing film 29 is tightened, and thus a cut surface is smooth and easy cutting is performed.

When the cut surface of the sealing film 29 is smooth, the downward flow of the preservation solution S may be smooth, which may improve the flow speed so as to shorten the use time of the preservation container 100 by medical staff for tests of the tissue T.

On the other hand, because the sealing film 29 deformed into the cap shape by the center post 34 has the inclined surface, as shown in FIG. 11C, a significant amount of the preservation solution S flows into the perimeter space 36 of the cutter holder 30, and flows down to the tissue accommodation portion 13 through the auxiliary through hole 32b.

In addition, the preservation solution S partially flows down to the tissue accommodation portion 13 through the space between the cutters 33 and through the through hole 32a in the center of the bottom surface 32.

Accordingly, the storage container 100 of the embodiment makes the cut surface of the sealing film 29 smooth and forms the auxiliary through hole 32b to make the movement speed of the preservation solution S faster.

FIGS. 14A to 14C show operating states of the storage container 100 according to another embodiment.

The storage container 100 of the embodiment differs from the above embodiment in that the cutter holder 30 includes the packing groove 32c, the seating portion 32d, the packing ring 32e, and the second locking protrusion 37, the seating projection 16 and the second stopper 17 are formed on the container body 10, and the skirt portion 29c is formed on the edge of the sealing film 29.

Other configurations are the same as above, and thus descriptions of the same parts will be omitted.

The storage container 100 of the embodiment is useful for protecting medical staff from the preservation solution S when collecting the tissue T in the laboratory.

Referring to FIGS. 14B and 14C, when the storage container 100 is turned upside down to collect the tissue T, the seating portion 32d of the cutter holder 30 is seated in surface contact with the seating projection 16 of the container body 10 and sealed by the packing ring 32e, which minimizes leakage of the preservation solution S between an outer circumferential surface of the cutter body 31 and an inner circumferential surface of the container body 10, thereby protecting the medical staff.

In addition, when the accommodation cap 24 and the holder body 31 are coupled to each other, the skirt portion 29c of the sealing film 29 is fitted between the accommodation cap 24 and the holder body 31.

The skirt portion 29c seals between the accommodation cap 24 and the holder body 31 to prevent the preservation solution S from leaking into the annular space 28, thereby protecting the medical staff.

The present invention's biological tissue storage container and method of using the same facilitate use when storing biological tissue in the storage container or collecting biological tissue from the storage container for examination, and can protect medical staff from leakage of a preservation solution such as formalin while using the storage container.

1. A biological tissue storage container configured for storing a biological tissue collected from an affected part while fixing the biological tissue in a preservation solution, the biological tissue storage container comprising:
a container body having an open upper portion and a closed lower portion, a first screw portion formed on an upper outer circumferential surface, a support projection formed on a lower side of the first screw portion, and a tissue accommodating portion formed to accommodate the biological tissue
a cap member comprising an upper surface portion a fastening cap extending downward from an edge of the upper surface portion and having a second screw portion formed on an inner circumferential surface and screw-coupled to the first screw portion an accommodation cap extending downward from the upper surface portion concentrically with the fastening cap on the inside of the fastening cap to form an accommodation space accommodating the preservation solution enabling entry into the inside of the container body, and having an open lower end, an annular space formed between the fastening cap and the accommodation cap and into which a holder body enters, and a sealing film that is breakable and adhered to a lower end of the accommodation cap to seal the accommodation space;
a cutter holder having the cylindrical holder body to which the accommodation cap is coupled inward, a bottom surface disposed at a lower end of the holder body and having a plurality of through holes formed therein, and a cutter formed on an upper surface of the bottom surface and breaking the sealing film;
a safety ring that is cylindrical and supported by the support projection, detachably coupled to the container body, and preventing breakage of the sealing film by the cutter by preventing the cap member from lowering; and
a coupling unit connecting the accommodation cap to the holder body to have a first frictional force to prevent the cutter holder from freely falling from the accommodation cap and at the same time, a second frictional force to enable relative movement of the accommodation cap with respect to the holder body.

2. The biological tissue storage container of claim 1, wherein
at least one of the accommodation cap and the holder body includes a synthetic resin material capable of elastic deformation, and the coupling unit includes a plurality of ribs are formed on an outer circumferential surface of the accommodation cap to protrude long vertically at regular intervals and at the same height, so that the accommodation cap and the holder body are fitted and coupled by the plurality of ribs.

3. The biological tissue storage container of claim 2, wherein
the sealing film includes a vinyl film heat-sealed at a lower end of the accommodation cap and a metal thin film bonded to the vinyl film,
a diameter of the sealing film is relatively larger than an outer diameter of the accommodation cap, so that a skirt portion protruding to the outside of the accommodation cap is formed, and
when the accommodation cap and the holder body are coupled to each other, the skirt portion of the sealing film seals between the accommodation cap and the holder body to prevent the preservation solution from leaking into the annular space.

4. The biological tissue storage container of claim 3, wherein
in the cutter holder, the cutter is formed in plural and arranged concentrically at a same height, and a center post relatively higher than a height of each cutter is formed in the center of the bottom surface, and a perimeter space into which the accommodation cap enters is formed between each cutter and the holder body, so that the sealing film is tightened by the center post just before the sealing film is broken by each cutter.

5. The biological tissue storage container of claim 4, wherein a plurality of auxiliary through holes are formed in a bottom surface of the perimeter space.

6. The biological tissue storage container of claim 1, wherein a plurality of first locking protrusions are formed on the bottom surface of the cutter holder on a same circumference,
an annular projection is disposed on an upper side of the tissue accommodating portion, protrudes from an inner wall surface of the container body, and supports the plurality of the first locking protrusions, and
at least one first stopper is formed to protrude on an upper surface of the annular projection such that each of the first locking protrusions is locked.

7. The biological tissue storage container of claim 1, wherein a plurality of second protrusions are formed to be spaced inward from an edge of the bottom surface of the cutter holder and disposed on a same circumference,
an annular seating projection is disposed on an upper side of the tissue accommodation portion to protrude from the inner wall surface of the container body, and on which a seating portion at an edge position of the bottom surface of the cutter holder is slidably seated, and
a plurality of second stoppers in which the second locking protrusions are locked are formed on the side of the annular seating projection at regular intervals.

8. The biological tissue storage container of claim 7, wherein an annular packing groove is formed in the seating portion of the bottom surface of the cutter holder, and a packing ring is coupled to the packing groove so as to be coplanar with the bottom surface of the cutter holder.

9. A method of using a biological tissue storage container of

6. the method comprising:
a first step, performed by a user, of holding the container body with one hand, holding the cap member with the other hand, and rotating and separating the cap member from the container body, wherein the cutter holder and the cap member while being coupled by the coupling unit are separated from each other;
a second step of placing the cutter holder and the cap member separated from the container body on a floor;
a third step of separating the safety ring from the container body with a thumb and index finger of the hand holding the container body;
a fourth step of accommodating the biological tissue collected from an affected part with the hand that held the cap member in the container body;
a fifth step of while holding the container body with one hand, screw-coupling the cap member and cutter holder to the container body with the other hand, breaking the sealing film with the cutter, and moving the preservation solution to the tissue accommodation portion in which the biological tissue is accommodated;
a sixth step of, after the fifth step, erecting the container body so that the cap member is in contact with the floor, moving the preservation solution of the tissue accommodation portion to an accommodation space of the cap member through the cutter holder, and then allowing the biological tissue to remain on the bottom surface of the cutter holder;
a seventh step of separating the container body from the cap member by holding the cap member with one hand, and rotating the container body with the other hand; and
an eighth step of holding the cap member with one hand, and separating the biological tissue from the cutter holder using tweezers with the other hand.

10. A method of using a biological tissue storage container of claim 8, the method comprising:
a first step, performed by a user, of holding the container body with one hand, holding the cap member with the other hand, and rotating and separating the cap member from the container body-(10), wherein the cutter holder and the cap member while being coupled by the coupling unit are separated from each other;
a second step of placing the cutter holder and the cap member separated from the container body on a floor;
a third step of separating the safety ring from the container body with a thumb and index finger of the hand holding the container body;
a fourth step of accommodating the biological tissue collected from an affected part with the hand that held the cap member in the container body;
a fifth step of while holding the container body with one hand, screw-coupling the cap member and cutter holder to the container body with the other hand, breaking the sealing film with the cutter, and moving the preservation solution to the tissue accommodation portion in which the biological tissue is accommodated;
a sixth step of, after the fifth step, erecting the container body so that the cap member is in contact with the floor, moving the preservation solution of the tissue accommodation portion to an accommodation space of the cap member through the cutter holder, and then allowing the biological tissue to remain on the bottom surface of the cutter holder;
a seventh step of separating the container body from the cap member by holding the cap member with one hand, and rotating the container body with the other hand; and
an eighth step of holding the cap member with one hand, and separating the biological tissue from the cutter holder using tweezers with the other hand.
